(19) United States
(12) Patent Application Publication
(10) Pub. No.: US 2026/0102597 A1
(43) Pub. Date: Apr. 16, 2026

(54) HEAD COVER FOR DISPENSING MEDICATION OR OTHER TREATMENT MATERIALS

(71) Applicant: Dazhai HUANG, Irvine, CA (US)

(72) Inventor: Dazhai HUANG, Irvine, CA (US)

(21) Appl. No.: 19/353,254

(22) Filed: Oct. 8, 2025

Related U.S. Application Data
(60) Provisional application No. 63/707,454, filed on Oct. 15, 2024.

(51) Int. Cl. A61M 35/00

(52) U.S. Cl. CPC A61M 35/10; A61M 2205/33; A61M 2210/04; A61M 2210/06

(57) ABSTRACT

This disclosure describes a head cover for dispensing medication, haircare product, or other treatment materials to a wearer's hair or scalp. The head cover includes a dome shaped head covering portion and multiple treatment material dispensing elements or patches mounted on its interior side. The patches may be pieces of permeable material permeated with the treatment material and capable of releasing the material over a period of time, or pouches containing the treatment material, or combinations thereof. Each patch may include a resistive heating element or a piezoelectric element to activate or accelerate the release of the treatment material. Control electronics may be provided to control the release based on patch locations, time, temperature, or other parameters. A reservoir containing the treatment material may also be provided to distribute the treatment material to the patches, and valves are provided on the connection tubes to control material release.

### BACKGROUND OF THE INVENTION

This invention relates to a head cover such as a hat or cap, and in particular, it relates to a head cover equipped with attachments for dispensing medication or other treatment materials to the wearer's scalp or hair.

### SUMMARY OF THE INVENTION

To achieve the above objects, the present invention provides a head cover for dispensing a treatment material to a wearer's hair or scalp, which includes a dome shaped head covering portion and a plurality of treatment material dispensing elements mounted on an interior side of the head covering portion.

In another aspect, the present invention provides a method for dispensing a treatment material to a wearer's hair or scalp, which includes providing a head cover having a dome shaped head covering portion and a plurality of treatment material dispensing elements mounted on an interior side of the head covering portion.

It is to be understood that both the foregoing general description and the following detailed description are exemplary and explanatory and are intended to provide further explanation of the invention as claimed.

### BRIEF DESCRIPTION OF DRAWINGS

FIG. 1 illustrates a hat in which embodiments of the present invention may be implemented.

FIG. 2 is a schematic bottom view showing a hat according to various embodiments of the present invention.

FIG. 3 is a schematic bottom view showing a hat according to various embodiments of the present invention.

FIG. 4 is a schematic side cross-sectional view showing a hat according to various embodiments of the present invention.

FIG. 5 is a schematic side cross-sectional view showing a hat according to various embodiments of the present invention.

### DETAILED DESCRIPTION OF THE INVENTION

Embodiments of the present invention provide a head cover, such as a hat or cap, which is equipped with attachments on its interior for dispensing medication or other treatment materials to the wearer's scalp or hair. The head cover may be made of any suitable material and have any style, e.g., with or without a brim or a visor or other related integrated parts such as neck flap, ear covers, etc. A hat with a visor is used in the descriptions below as an example, but the invention is not limited to such.

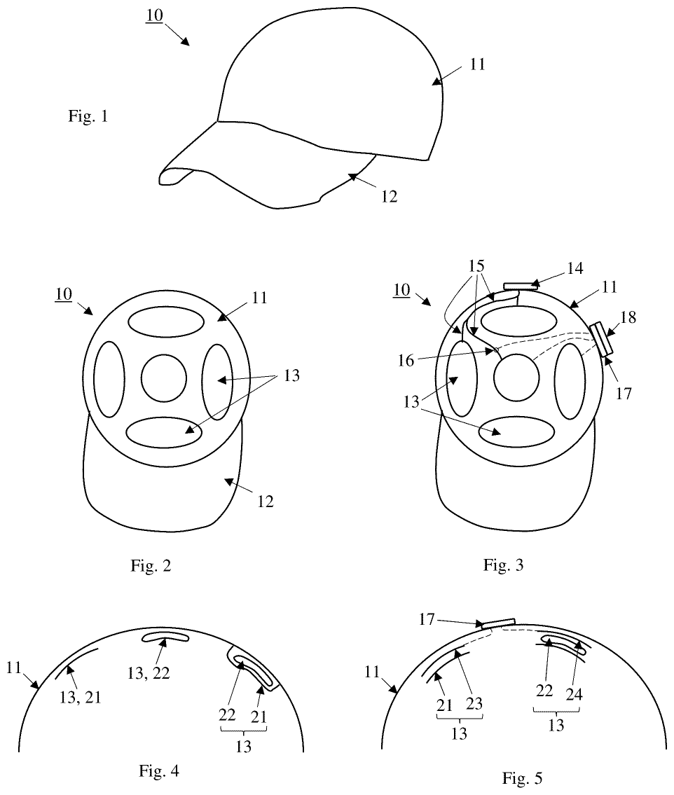

FIG. 1 illustrates the exterior appearance of hat 10, with a dome shaped head covering portion 11 and a visor 12 in this example. FIG. 2 illustrates the interior of the hat 10 viewed from the underside. The hat is provided with one or more dispensing elements 13 that are attached to various locations on the interior of the head covering portion 11 for dispensing medication, haircare products, skincare products, or other treatment or care products (collectively, treatment materials) to the hair or the scalp of the wearer. The dispensing elements 13 are preferably thin and flexible in form. For convenience, these dispensing elements are referred to as patches in the descriptions below.

When the hat is worn by a wearer, the patches are in direct contact with the wearer's hair or scalp. The patches are preferably located at various selected locations of the hat, such as the front, rear, side, top, etc. portions of the hat. The locations of the patches are preferably specific to the locations of the conditions of the wearer that is being treated. In some embodiments, as described in more detail below, the interior of the hat is provided with multiple attaching structures at various locations, and the patches can be selectively attached to desired locations based on the wearer's choice. Different patches may contain different treatment materials.

The treatment materials may have any suitable chemical nature and physical form, such as liquid, cream, gel, dried liquid or cream or gel, solid, etc. The physical form may affect the manner it is released from the patches.

Preferably, as described in more detail below, control mechanisms are provided to control the release of the treatment materials from the patches. The control mechanism may be configured to control the release based on the patch locations, time, temperature, and other parameters.

Preferably, the treatment material dispensing system, including the patches and any control devices, is detachably mounted on the hat. Various components are replaceable (e.g. when the treatment material is exhausted). The entire treatment material dispensing system is supported on the hat itself, and may be removed from the hat. It should be noted that the invention does not pertain to a larger hair or head treatment device that is required to be attached to other fixtures or equipment and/or plugged into a power receptacle during normal operation.

Many specific structures may be used to implement the hat with a treatment material dispensing system according to embodiments of the present invention, as schematically illustrated in FIGS. 3-5. It should be noted that these figures contain schematic drawings which do not represent the precise shapes, sizes and/or locations of the various components. In particular, in FIGS. 4 and 5, various connected components are shown as being separate from each other and the gaps between them are exaggerated for purposes of clarity of illustration.

In some embodiments (see FIG. 4), each patch 13 is made of a flexible piece of permeable material 21, such as a fabric material, attached to the hat. The permeable material 21 is permeated (e.g. pre-soaked or otherwise treated) with the treatment material and is capable of releasing the treatment material. In some examples, the treatment material is slowly released over a period of time, e.g., over the course of a few hours, a day, or a few days, etc. In some examples, the release of the treatment material is initiated or accelerated by heat or pressure. For example, the release may be initiated or accelerated when the hat is worn by a wearer and the patch is exposed to body temperature. Or, the release may be initiated or accelerated by a pressure manually applied by the wearer.

In some embodiments (see FIG. 4), the patch 13 includes a flexible pouch 22 that contains the treatment material, and the material may be released from the pouch by pressure, gravity, capillary action, heat, or other physical mechanisms. The pouch may have a pressure sensitive valve, an electrically controlled valve, micro holes, a wick, or other openings or structures to release the treatment material. In some examples, the treatment material is slowly released over a period of time, e.g., over the course of a few hours, a day, or a few days, etc. In other examples, the release of the treatment material is initiated or accelerated by heat (e.g. exposure to body temperature) or pressure (e.g. manually applied pressure). (Again, for purposes of clarity of illustration, FIG. 4 shows the pouch 22 as being separated from the hat 11.)

In some embodiments (see FIG. 4), the patch 13 is a combination of pieces of a permeable or wicking material 21 (e.g. fabric) and a pouch 22. For example, it may be a pocket formed by the permeable or wicking material (at least on the side that contacts the wearer), with a pouch containing the treatment material placed in the pocket, and the treatment material can be released from the pouch and permeates the permeable material. In another example, the pouch has a piece of wicking material adhered to its outside on the side that contacts the wearer's hair or scalp.

In some embodiments (see FIG. 3), in lieu of a pouch in each patch 13, a reservoir 14 containing the treatment material is attached to the hat, e.g. to the visor or brim or other suitable locations not directly in contact with the wearer's head (to avoid discomfort), and flexible tubes 15 are provided to distribute the treatment material from the reservoir to the patches 13 (e.g., a piece of permeable or wicking material). The reservoir may be a head shelled container (e.g. a vial) or a flexible container. A valve 16 may be provided on the tube associated with each patch, and/or a main valve may be provided on a tube that leads from the reservoir to the multiple branching tubes.

In some embodiments (see FIG. 5), the patch 13 contains a heating element 23, such as a resistive heating element (e.g., a thin substrate with thin heating wires arranged on it), and control electronics 17 (e.g. a processor chip) that control the heating element to heat the treatment material to release it from the pouch or the permeable or wicking material in a controlled manner.

In some embodiments (see FIG. 5), the patch 13 contains a pressure generating element 24, such as a piezoelectric element, and control electronics 17 that control the pressure generating element to eject the treatment material from the pouch 22 in a controlled manner.

In some embodiments, the pouch 22 or the reservoir 14 is internally pressurized, and valves on the pouch or the reservoir, or the valves on the distribution tubes connecting the reservoir to each patch, are controlled by control electronics 17 to open and close to control the release the treatment material.

The control electronics 17 are also shown in FIG. 3, along with dashed lines representing electrical signal lines. In the cases where control electronics and other relevant electrical structures (heating element, pressure generating element, valve, etc.) are used, a power source 18 such as a battery is provided to power the control electronics and other electrical structures (see FIG. 3). The control electronics 17 and battery 18 may be attached to the hat at any suitable locations.

When control electronics are used, they may be programmed to control the release of the treatment material based on time (in which case a timer is provided in the electronics), temperature (in which case a temperature sensor is provided in the electronics), or other parameters. For example, the release may controller to occur at specified times of the day, periodically at specified intervals, etc. The control electronics may also be programmed to control the amount of the treatment material released, e.g., by controlling the amount of heat or pressure generated, or the opening size of the valve, etc.

In some embodiments, the interior of the hat is provided with multiple attaching structures at various locations, and the patches may be selectively attached to some or all of these locations based on the wearer's choice. The attaching structures may be, for example, hoop-and-loop fasteners, pockets, belts, hooks, snaps, pins, adhesive materials, and/or any other suitable structures. If a reservoir is used to supply the treatment materials, the tubes may be permanently or detachably mounted on the hat. If control electronics are used, the electrical wires may be permanently or detachably mounted on the hat. The reservoir and the control electronics including batteries may be mounted on the hat at any suitable locations by any suitable structure.

To summarize, the dispending elements of the head cover may include combinations and permutations of any treatment material, any physical forms described above, any attaching structures described above, any spatial distribution on the head cover, and any material releasing methods described above including passive and active release methods and with or without electronic control.

It will be apparent to those skilled in the art that various modification and variations can be made in the head cover for dispensing treatment materials of the present invention without departing from the spirit or scope of the invention. Thus, it is intended that the present invention cover modifications and variations that come within the scope of the appended claims and their equivalents.

1. A head cover for dispensing a treatment material to a wearer's hair or scalp, comprising:
a dome shaped head covering portion; and
a plurality of treatment material dispensing elements mounted on an interior side of the head covering portion at selected locations, configured to release a treatment material.

2. The head cover of claim 1, wherein each dispensing element includes a piece of flexible and permeable material which is permeated with the treatment material and is capable of releasing the treatment material over a period of time.

3. The head cover of claim 1, wherein each dispensing element includes a pouch containing the treatment material.

4. The head cover of claim 1, wherein each dispensing element includes a resistive heating element or a piezoelectric element configured to activate or accelerate release of the treatment material.

5. The head cover of claim 4, further comprising control electronics coupled to the resistive heating element or the piezoelectric element to control the release of the treatment material.

6. The head cover of claim 5, wherein the control electronics is control electronics are configured to control the release of the treatment material based on patch locations, time, and/or temperature.

7. The head cover of claim 1, wherein each dispensing element includes a piece of flexible and permeable material, the head cover further comprising a reservoir containing the treatment material, and a plurality of tubes with valves connecting the reservoir to the dispensing elements.

8. The head cover of claim 7, further comprising control electronics coupled to the valves to control the release of the treatment material.

9. The head cover of claim 8, wherein the control electronics is control electronics are configured to control the release of the treatment material based on the patch locations, time, and/or temperature.

10. The head cover of claim 1, wherein the treatment material dispensing elements are detachably mounted on the head covering portion.
